(19) United States
(12) Patent Application Publication
(10) Pub. No.: US 2025/0235507 A1
(43) Pub. Date: Jul. 24, 2025

(54) INHALABLE POROUS MICROSPHERE LOADED WITH RECOMBINANT HUMAN RELAXIN-2 AND PREPARATION METHOD THEREFOR

(71) Applicant: SHANDONG UNIVERSITY, Jinan (CN)

(72) Inventors: Guimei LIN, Jinan (CN); Shengnan QIU, Jinan (CN); Xianglei FU, Jinan (CN); Xiaogang ZHAO, Jinan (CN)

(21) Appl. No.: 19/175,639

(22) Filed: Apr. 10, 2025

Related U.S. Application Data
(63) Continuation of application No. PCT/CN2023/135790, filed on Dec. 1, 2023.

(30) Foreign Application Priority Data
Feb. 3, 2023 (CN) 202310054616.6

(51) Int. Cl. A61K 38/22; A61K 9/00; A61K 9/16

(52) U.S. Cl. CPC A61K 38/2221; A61K 9/0075; A61K 9/1635; A61K 9/1658; A61K 9/1694

(57) ABSTRACT

The invention relates to an inhalable porous microsphere loaded with recombinant human relaxin-2 and a preparation method therefor, and belongs to the technical field of biological medicines. The preparation method comprises the steps of: (1) dissolving recombinant human relaxin-2 in PBS containing bovine serum albumin to serve as an internal aqueous phase, and dissolving a carrier material ester-terminated polylactic acid copolymer OH-PLGA75/25-COOR in dichloromethane to serve as an oil phase; preparing a W1/O emulsion; (2) injecting the W1/O emulsion obtained in step (1) into a PVA solution to prepare non-porous microspheres loaded with RLX (RLX@SMs); and (3) preparing the porous microspheres loaded with RLX from the RLX@SMs obtained in step (2) by a programmed cooling method. The RLX@PMs obtained have a larger geometric diameter, can release drugs for a long time, and has a smaller aerodynamic diameter, which is beneficial for high deposition in the deep part of the lung.

### FIELD OF THE INVENTION

The invention relates to an inhalable porous microsphere loaded with recombinant human relaxin-2 and a preparation method therefor, and belongs to the technical field of biological medicines.

### BACKGROUND OF THE INVENTION

Idiopathic pulmonary fibrosis (IPF) causes progressive deterioration of lung function, and there is currently no effective treatment for this disease etiology. Recombinant human relaxin-2 (RLX) is a polypeptide hormone discovered in the early 20th century that is secreted in large quantities during late-stage pregnancy and has a relaxing effect on the birth canal. It can play a role in dilating blood vessels, inducing the degradation of extracellular matrix and reversing the activation of fibroblasts in various tissues and organs, thus inhibiting the development of fibrosis. It is a promising biotherapeutic candidate drug for anti-structural remodeling and anti-pulmonary fibrosis. However, because it is a protein polypeptide drug with a very short plasma half-life (about 10 minutes), it rarely reaches the target organ to exert its efficacy after intravenous injection. To achieve optimal therapeutic efficacy, continuous infusion or repeated injection is required.

### SUMMARY OF THE INVENTION

In view of the above problems, the invention provides an inhalable porous microsphere loaded with recombinant human relaxin-2 (RLX) and a preparation method therefor. The invention evaluates the therapeutic effect of porous microspheres loaded with RLX (RLX@PMs) on IPF through inhalation administration. RLX@PMs exhibits a larger geometric diameter as sustained-release RLX reservoirs. However, due to their porous structure, they have a smaller aerodynamic diameter, which is beneficial for high deposition in the deep part of the lung. Experimental results demonstrate prolonged drug release over 24 days, with released RLX retaining its peptide structure and activity. The technical solutions of the invention are as follows.

A preparation method for inhalable porous microspheres loaded with recombinant human relaxin-2, comprising the steps of:
(1) dissolving recombinant human relaxin-2 (RLX) in phosphate-buffered saline (PBS) containing bovine serum albumin (BSA) to serve as an internal aqueous phase, and dissolving a carrier material ester-terminated polylactic acid copolymer OH-PLGA75/25-COOR in dichloromethane (DCM) to serve as an oil phase; adding the internal aqueous phase into the oil phase and emulsifying by high-speed shearing at 10,000 rpm for 2 minutes to form a W1/O emulsion; using 20% (v/v) polyvinyl alcohol (PVA) solution as an external aqueous phase;
(2) injecting the water-in-oil (W1/O) emulsion obtained in step (1) into a 20% (v/v) PVA solution pre-cooled in an ice bath, mixing and emulsifying for 2 minutes by using high-speed shearing force to form an oil-in-water (O/W2) emulsion, stirring the emulsion in an ice bath to evaporate DCM, collecting non-porous microspheres by centrifuging at a speed of 5,000-8,000 rpm for 5-8 minutes, washing the microspheres with deionized water pre-cooled in an ice bath, and collecting all precipitates to obtain non-porous microspheres loaded with RLX (RLX@SMs); and
(3) preparing the porous microspheres loaded with recombinant human relaxin-2 (RLX@PMs) from the non-porous microspheres loaded with RLX obtained in step (2) by programmed cooling which specifically comprises the steps of: first, freezing the collected non-porous microspheres at −20° C. for 4-6 hours, then immediately transferring them to a −80° C. freezer and maintaining for 8 hours. and finally lyophilizing the microspheres in a freeze dryer for 24 hours to obtain the porous microspheres.

Preferably, in step (1), the recombinant human relaxin-2 (RLX) is dissolved in PBS containing 0.1% (v/w) of bovine serum albumin (BSA).

Preferably, in step (1), the ester-terminated polylactic acid copolymer OH-PLGA75/25-COOR has a lactide-to-glycolide ratio (LA:GA) of 75:25 and a molecular weight (Mw) of 28,500 Da.

Preferably, in step (1), the carrier material ester-terminated polylactic acid copolymer OH-PLGA75/25-COOR is dissolved in DCM at a concentration of 100 mg/mL to serve as the oil phase.

Preferably, in step (2), the oil-in-water (O/W2) emulsion is stirred in ice water to evaporate DCM, and the nonporous microspheres are collected by centrifugation at 8,000 rpm for 5 minutes.

Preferably, in step (2), the collected non-porous microspheres are washed three times with deionized water pre-cooled in an ice bath.

Preferably, in step (3), the collected non-porous microspheres are first frozen at −20° C. for 4 h.

The present invention further comprises inhalable porous microspheres loaded with recombinant human relaxin-2 prepared by the above method. The particle size of the microspheres is 8-10 μm, and the particle density and porosity are 0.199-0.041 g·cm⁻³ and 95.0±0.8%, respectively.

Compared to the prior art, the invention exhibits the following advantages:

(1) The re-emulsification method combined with gradient cooling technology adopted by the invention is a simple, ultra-fast, low-cost and environment-friendly method for preparing porous microspheres loaded with protein polypeptide drugs, which can avoid the use of pore-forming agents, thereby avoiding the potential safety hazard caused by residual pore-forming agents in the industrial-scale production of porous microspheres. The inhalable porous microspheres obtained by the invention exhibit a larger geometric diameter with particle size of 8-10 μm. They can be used as RLX reservoirs for a long-term drug release. They have a smaller aerodynamic diameter due to their porous structures, which is beneficial for high deposition in the deep part of the lung. Also, they have excellent protein loading efficiency, with drug loading of 0.89±0.22% and encapsulation efficiency of 82.23±3.75%. Their particle density and porosity are 0.199±0.041 g·cm⁻³ and 95.0±0.8%, respectively. The low density and high porosity are beneficial for the pulmonary deposition of microspheres after inhalation administration.

(2) In vitro drug release studies demonstrated sustained RLX release from the porous microspheres for over 24 days, with no significant initial burst release. The biological distribution of the porous microspheres in the lung showed that their sustained release effect lasted for at least 1.5 month. The low particle density of the porous microspheres allows deep lung penetration, ensuring more effective deposition at the target site. Compared with the free drug without carrier protection, they will not be quickly eliminated from the lung. On the contrary, non-porous microspheres predominantly deposit in the stomach rather than the respiratory tract.

(3) In a bleomycin-induced pulmonary fibrosis mouse model, histopathological analysis showed that RLX@PMs alleviated the effects of excessive collagen deposition, structural distortion and decreased compliance in the lung tissue of mice after a single inhalation. Inhalation of RLX@PMs in each group of mice can significantly enhance the recovery of lung function after bleomycin-induced lung injury, and can improve lung function, thus increasing the survival period. In addition, RLX@PMs showed better safety than frequent administration of pirfenidone. Therefore, the slow release of RLX with porous microspheres as carriers in the deep part of the lung has great potential in the effective treatment of pulmonary fibrosis.

### BRIEF DESCRIPTION OF THE DRAWINGS

FIG. 1 shows dried microsphere powder and suspension after re-suspension of non-porous (a) and porous (b) microspheres; the SEM images, and bright field and rhodamine B fluorescence field images of the two microspheres, with scale of 10 microns.

FIGS. 2A-B show the cumulative release curves of model protein BSA loaded in the non-porous (a) and porous (b) microsphere preparations;
the data are mean±SD, with n=3 at each time point;

FIG. 3 shows in vivo imaging pictures of the porous microspheres loaded with rhodamine B retained in the airway of mice within 4 to 48 days after a single inhalation;

FIG. 4 shows the results of in vivo high-resolution micro-CT scanning of lung tissues of mice in each group to monitor the protective effect of RLX@PMs on BLM-induced pulmonary fibrosis at different treatment time points;

FIG. 5 shows the curves of weight change of mice in each treatment group; all quantitative data are mean±SD (n=5);

FIG. 6 shows the lung coefficients of mice after 4 weeks of drug intervention in all the groups. All quantitative data are mean±SD (n=5);

FIG. 7 shows the histopathological analysis of the lung of mice treated with different drugs for several weeks; where in 7a, hematoxylin and eosin staining are adopted; in 7b, Masson staining is adopted, to determine the changes of collagen deposition in the lung tissue, with scale of 100 microns;

FIG. 8 shows images taken by a polarizing microscope (400× magnification) of the lung tissue in each treatment group after Sirius red staining;

FIG. 9 shows the quantitative analysis of the deposition change of type I collagen in the lung tissue after Mason staining, n=3 animals/group; and

FIG. 10 shows the quantitative analysis of collagen after Sirius red staining, n=3 animals/group.

### DETAILED DESCRIPTION OF THE INVENTION

The invention is further described in conjunction with specific examples below, through which the advantages and characteristics of the invention will become apparent. However, these examples are provided for illustrative purposes only and shall not be construed as limiting the scope of the invention in any manner. It should be understood by those skilled in the art that the details and forms of the technical solution of the present invention can be modified or substituted without departing from the spirit and scope of the invention, and these modifications and substitutions are all within the scope of protection of the invention.

### Example 1: An Inhalable Porous Microsphere Loaded with Recombinant Human Relaxin-2 and Preparation Method Therefor

(1) The recombinant human relaxin-2 (RLX) was dissolved in 0.2 ml of PBS containing 0.1% (v/w) of bovine serum albumin (BSA) to serve as an internal aqueous phase. A certain amount of carrier material ester-terminated polylactic acid copolymer OH-PLGA75/25-COOR (LA:GA=75:25, molecular weight (Mw) of 28,500 Da) was dissolved in 1 mL of dichloromethane (DCM) at a concentration of 100 mg/mL to serve as an oil phase. The internal aqueous phase was added to the oil phase, and a water-in-oil (W1/O) emulsion was formed after high-speed shearing at the speed of 10,000 rpm for 2 minutes. A 20% (v/v) polyvinyl alcohol (PVA) solution was prepared as an external aqueous phase.

(2) The water-in-oil (W1/O) emulsion obtained in step (1) was injected into 5 mL of 20% (v/v) PVA solution pre-cooled in an ice bath, mixed and emulsified for 2 minutes with high-speed shearing force to form an oil-in-water (O/W2) emulsion. The emulsion was stirred in ice water for 4 h to volatilize DCM. The microspheres were collected by centrifugation at 8,000 rpm for 5 minutes, washed three times using deionized water pre-cooled in an ice bath, and all the precipitates were collected. Then, porous microspheres loaded with recombinant human relaxin-2 (RLX@PMs) were prepared by programmed cooling, which specifically comprises the steps of: first, freezing the collected non-porous microspheres at −20° C. for 4 hours, then immediately transferring them to a refrigerator at −80° C. for 8 hours, and finally subjecting them to freeze-drying for 24 hours, thereby obtaining the porous microspheres.

### Example 2: An Inhalable Porous Microsphere Loaded with Recombinant Human Relaxin-2 and Preparation Method Therefor

(1) The same as the step (1) in Example 1.

(2) The water-in-oil (W1/O) emulsion obtained in step (1) was injected into a 20% (v/v) PVA solution pre-cooled in an ice bath, mixed and emulsified with high-speed shearing force for 2 minutes to form an oil-in-water (O/W2) emulsion. The emulsion was stirred in ice water to evaporate DCM, centrifuged at 6500 rpm for 7 minutes to collect non-porous microspheres. The microspheres were washed with deionized water pre-cooled in an ice bath, and all the precipitates were collected, thereby obtaining non-porous microspheres loaded with RLX (RLX@SMs).

(3) The porous microspheres loaded with recombinant human relaxin-2 (RLX@PMs) were prepared from the non-porous microspheres loaded with RLX obtained in step (2) by programmed cooling, which specifically comprises the steps of: first, freezing the collected non-porous microspheres at −20° C. for 5 hours, then immediately transferring them to a refrigerator at −80° C. for 8 hours, and finally transferring them to a freeze dryer for freeze drying for 24 hours, thereby obtaining the porous microspheres.

### Example 3: An Inhalable Porous Microsphere Loaded with Recombinant Human Relaxin-2 and Preparation Method Therefor

(1) The same as the step (1) in Example 1.

(2) The water-in-oil (W1/O) emulsion obtained in step (1) was injected into a 20% (v/v) PVA solution pre-cooled in an ice bath, mixed and emulsified with high-speed shear force for 2 minutes to form an oil-in-water (O/W2) emulsion. The emulsion was stirred in ice water to evaporate DCM, centrifuged at 5,000 rpm for 8 minutes to collect non-porous microspheres. The microspheres were washed with deionized water pre-cooled in an ice bath, and all the precipitates were collected, thereby obtaining non-porous microspheres loaded with RLX (RLX@SMs).

(3) The porous microspheres loaded with recombinant human relaxin-2 (RLX@PMs) were prepared from the non-porous microspheres loaded with RLX obtained in step (2) by programmed cooling, which specifically comprises the steps of: first, freezing the collected non-porous microspheres at −20° C. for 6 h, then immediately transferring them to a refrigerator at −80° C. for 8 h, and finally transferring them to a freeze dryer for freeze drying for 24 h, thereby obtaining the porous microspheres.

### Experimental Example 1: Characterization of Inhalable Porous Microspheres Loaded with Recombinant Human Relaxin-2 Obtained by the Invention

### 1.1 Structural Characterization

The freeze-dried non-porous and porous microspheres were examined. The RLX@PMs microspheres were re-suspended in a solution according to the manufacturer's instructions, and the particle size distribution was analyzed using an ultra-depth three-dimensional microscope. The freeze-dried microspheres were coated on the conductive adhesive, and the morphology of microspheres was observed by focused ion beam-energy dispersive X-ray spectroscopy-scanning electron microscope (FIB-EDS-FIB).

### 1.2 Determination of Porosity and Density of Porous Microspheres

The porosity and density of porous microspheres were determined using an ethanol infiltration method. A certain amount of freeze-dried porous microspheres was weighed, immersed in 5 mL of anhydrous ethanol, and ultrasonically treated for 10 minutes, to allow anhydrous ethanol to completely penetrate the pores of the porous microspheres. Additional anhydrous ethanol was then added to reach the initial volume of 5 mL. The weight of the lyophilized RLX@PMs was recorded as m1, and the weight of the tube filled with absolute ethanol was recorded as m2. The total weight of RLX@PMs and anhydrous ethanol after ultrasonic treatment was recorded as m3. The total weight after adding anhydrous ethanol was recorded as m4. The weight of wet RLX@PMs with ethanol absorbed was denoted as m5. The density of anhydrous ethanol is ρₑ.

The porosity (P) of RLX@PMs is calculated as follows:

$${P}_{RLX @ PMs} ( \% ) = ( m 5 - m 2 ) / [ m 3 - ( m 4 - m 5 ) - m 2 ] * 100 \% .$$

The density of porous microspheres is calculated as follows:

$${ρ}_{RLX @ PMs} = m 1 * {ρ}_{e} / ( m 3 + m 5 - m 4 - m 2 ) .$$

### 1.3 Drug Loading and Encapsulation Efficiency

After the microspheres were washed, the supernatant and all the washing liquid were collected by centrifugation, and the drug content in the solution was calculated. The drug content in the supernatant and washing liquid was determined by BCA protein concentration detection method using ELISA kit. Then, the encapsulation efficiency (EE) and drug loading (DL) were calculated by the following formula.

$$EE \% = {W}_{encapsulated BSA} / {W}_{total BSA} \times 100 \% .$$

$$DL \% = {W}_{encapsulated BSA} / {W}_{RLX @ PMs} \times 100 \% .$$

### 1.4 In Vitro Release Behavior

RLX was not easily detected in the long-term release experiment because of its low content and slow release, and bovine serum albumin (BSA) was used as one of the most stable and widely used model proteins for in vitro release experiments of long-term sustained-release delivery systems. Therefore, in this experiment, we monitored the release behavior of BSA. BSA itself is also a protein stabilizer for preparing RLX-loaded microspheres. The process of preparing porous microspheres loaded with BSA was exactly the same as that of loading RLX. Non-porous or porous microspheres (50 mg) loaded with the model protein BSA were put into a dialysis bag (molecular weight cut-off of 28.5 kDa) together with 2 mL of a release medium and suspended in a 30 mL centrifuge tube. The release medium was phosphate buffered saline (PBS, pH 7.4, 37° C.). The test tube was oscillated at a speed of 100 rpm in a thermostat gas bath vibrator. At the required time interval, 2 mL of the release medium was taken out, and then an equal volume of fresh medium was added. The released samples were stored in the refrigerator until they were quantified by an ELISA kit.

### 1.5 Experimental Results

### 1.5.1 Characterization of Microspheres

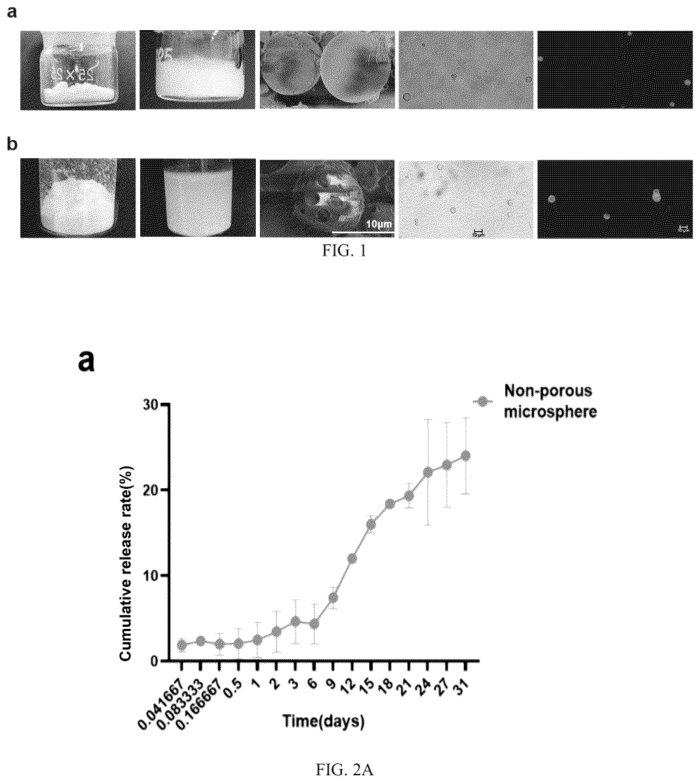

Non-porous PLGA microspheres were formed by the water-in-oil-in-water (W1/O/W2) method. Porous PLGA microspheres were generated by subjecting these microspheres to gradient cooling, followed by freeze-drying In contrast, the non-porous microspheres prepared by the same double emulsification method were directly freeze-dried after evaporation of solvent without programmed cooling to obtain white powder (FIG. 1a). Freeze-drying of porous microspheres also provided white fluffy powder with superior resuspension properties compared to non-porous microspheres. The morphology of the two kinds of microspheres was observed by SEM, and the images could show the morphology and dispersity of the prepared non-porous and porous microspheres. As shown in FIG. 1b, RLX@PMs have a porous spherical structure with a round shape and uniform size distribution, mostly between 8-10 μm. The larger geometric size of the microspheres can greatly prolong their residence time in the lung. In addition, the porous microspheres loaded with fluorescent dye (rhodamine B) showed that these microspheres have an obvious, uniform and compact pore structure, which may be beneficial for the loading and delivery of recombinant human relaxin-2. According to the method described in the manufacturer's instructions, the loading efficiency of PLGA microspheres was verified by using BSA as the model protein. The drug loading of the PLGA microspheres was 0.89±0.22% and the encapsulation efficiency was 82.23±3.75%. The particle density and porosity of the microspheres were 0.199±0.041 g·cm⁻³ and 95.0±0.8%, respectively. These observations confirmed that the gradient cooling and freezing technology (without pore-forming agent) was an efficient and environmentally friendly method for porous microspheres fabrication.

### 1.5.2 Investigation on In Vitro Release Behavior of Microspheres

The in vitro release efficiency of non-porous and porous microspheres was studied in phosphate buffered saline (pH 7.4) at 37° C. In vitro drug release curves (FIGS. 2a and 2b) showed that the cumulative percentage of drug release from the drug-loaded porous and non-porous microspheres was time-dependent. It was clear that the release time of drugs loaded in the porous microspheres can be as long as more than 24 days, and there was no obvious initial explosive release. However, the cumulative release rate of BSA loaded in the non-porous microspheres was only 24% over the same release time, so it could be predicted that its release process would last longer.

### Experimental Example 2: In Vivo Study

### 2.1 Establishment of a Mouse Model Induced by Bleomycin

C57BL/6J mice (20±2 g, female) were purchased from Beijing Life River Experimental Animal Technology Co., Ltd, and were housed in specific pathogen-free animal facilities. For the induction of fibrosis, mice were treated with 100 μL of bleomycin (with concentration of 1 mg/mL) at a dose of 5 mg/Kg via tracheal intubation.

### 2.2 Evaluation of Biological Distribution and Retention Time of Porous Microspheres in Vivo

In order to evaluate the biological distribution and retention time, rhodamine B labeled non-porous PLGA microspheres (rhodamine B@SMs) or porous PLGA microspheres (rhodamine B@PMs) were inhaled through nebulization, and rhodamine B-free was used as control. Real-time fluorescence images were obtained by the small animal in vivo imaging system at regular intervals. The same treatment was carried out using the same dose of rhodamine B-free as the control group.

### 2.3 Evaluation of the Effect of Anti-Fibrosis Ability

After two weeks of bleomycin administration, mice were divided into four groups. In the RLX@PMs group, porous microspheres loaded with RLX were administered at a single dose of 70 μg/mouse (including RLX) to each mouse via nebulization. Similarly, in the RLX-free group, the RLX-free solution was administered via nebulization at a dose of 5 μg/mouse daily for two weeks. In addition, a blank control group only nebulized with PBS (PBS group) was also set up. Pirfenidone was used as a positive control, and was administered via intragastric gavage to mice at a dose of 300 mg/kg daily for two weeks (PFD group). The weight of mice was recorded once every two days.

For further examination, after anesthesia, whole-lung imaging was performed using a micro-computed tomography (micro-CT) imaging system for small animals (scanning parameters: scan resolution, 18 μm; voltage, 70 kV; current, 100 μA). After treatment, all mice were euthanized and tissues were collected for further analysis, and lung coefficient was calculated by the wet weight of lungs.

### 2.4 Hematoxylin and Eosin Staining, Mason Trichrome Staining and Sirius Red Staining

In addition to in vivo imaging examination, we also used hematoxylin and eosin (H&E) staining, Masson's trichrome staining and Sirius red staining to detect the histological changes and collagen deposition in the lungs according to the manufacturer's instructions. After the mice were euthanized, the whole lung tissues were collected and fixed in 4% paraformaldehyde overnight. Subsequently, the tissues were subjected to gradient dehydration according to the standard procedure and embedded with paraffin. The lung tissues of mice were embedded in paraffin and cut into 3 μm sections, followed by H&E staining. Staining was carried out according to the operating instructions of a Masson trichrome staining kit. The blue collagen tissues were visible under an optical microscope. The stained sections were imaged using a VS120 virtual slide microscope, and the severity of interstitial fibrosis was evaluated at 100× magnification by measuring the percentage of blue stained area. In addition, Sirius red staining was performed by using a Sirius red staining kit. After staining, the stained sections were scanned by a polarization microscope, and the degree of collagen deposition in the lung was evaluated based on the image. All collagen density data were quantified by ImageJ software.

### 2.5 Experimental Results

### 2.5.1 Deposition of Porous Microspheres in Mouse Lung

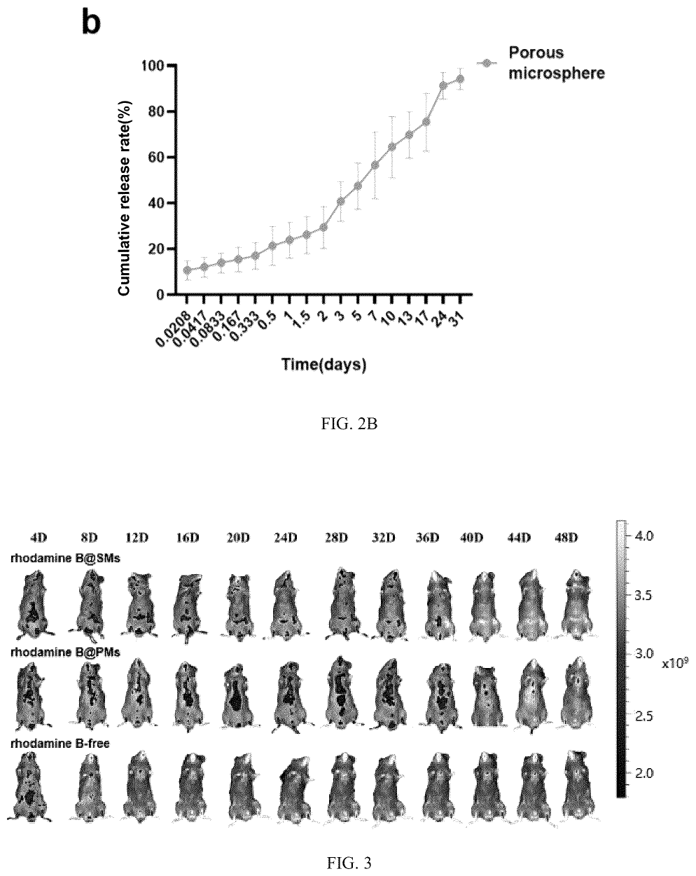

The deposition of rhodamine B@PMs in the lung tissue was studied by in vivo imaging of near infrared ray (NIR) dye (rhodamine B). We found that rhodamine B@PMs were deposited more in the gastrointestinal tract than in the lung after administration. In contrast, the proportion of fluorescence signals of rhodamine B@PMs and free dyes that were deposited in the lung was higher, as shown in FIG. 3. It was speculated that the actual density of non-porous microspheres was too high, which led to many microspheres entering the lower respiratory tract. The fluorescence signals of rhodamine B-free in the free dye group disappeared completely within 2 weeks, indicating that the free drug without carrier protection would be quickly eliminated from the lung. In contrast, the slow-release effect of rhodamine B@PMs lasted at least 1.5 months, which was why the microspheres had a significant sustained-release effect.

### 2.5.2 Anti-Fibrosis Effect of RLX@PMs In Vivo

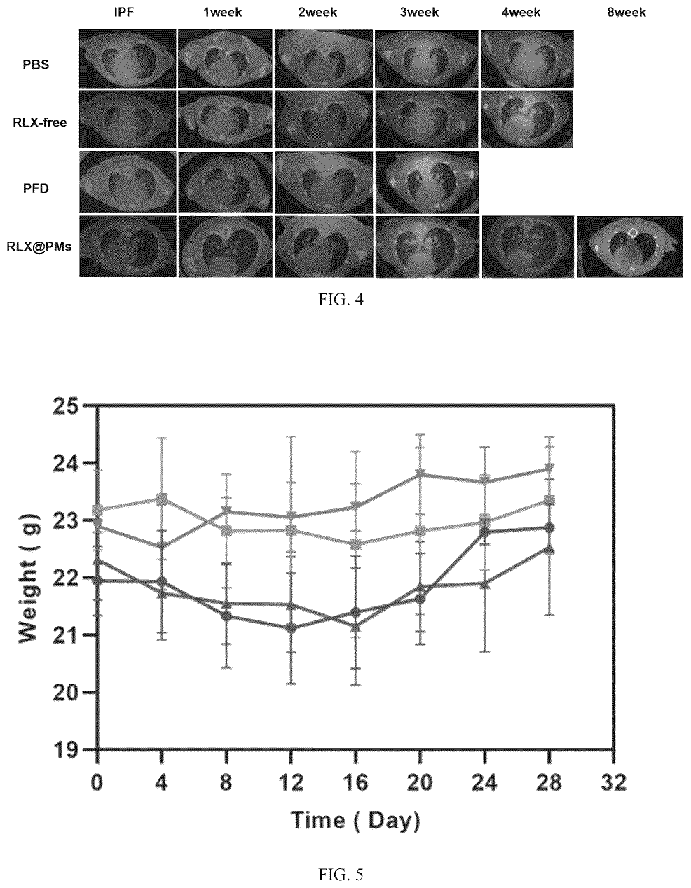

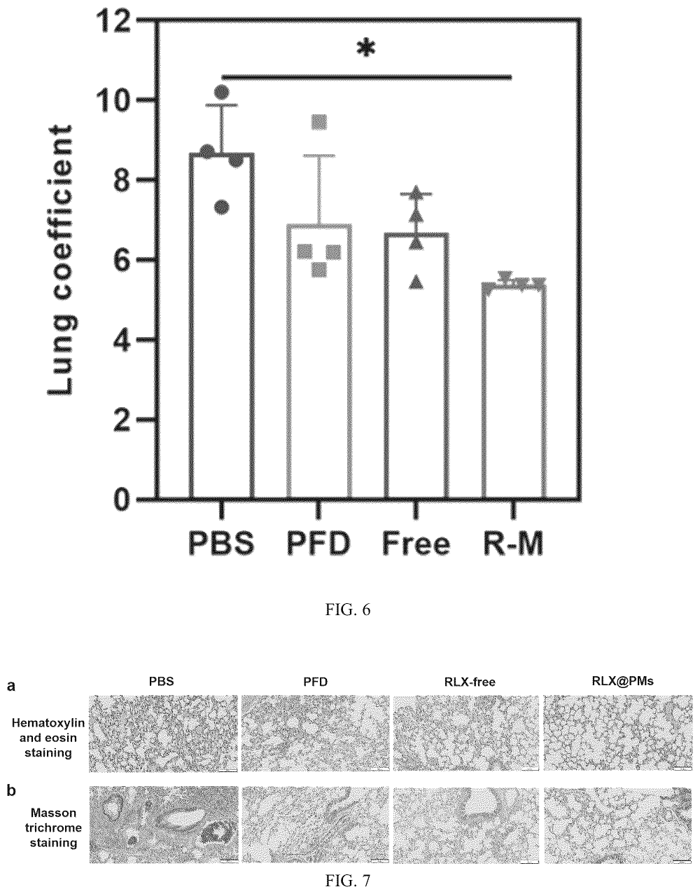

Next, the anti-fibrosis ability of RLX@PMs in a bleomycin-induced pulmonary fibrosis mouse model was further evaluated. All animals were dosed regularly, and the degree of pulmonary fibrosis in mice was examined by imaging. Micro-CT scan showed that the induction of bleomycin led to obvious changes in lung structure, with extensive pulmonary fibrosis changes occurring in the lungs of mice at week 2. After 4 weeks of treatment, as shown in FIG. 4, the PBS group showed progressive severe pulmonary fibrosis, the positive control drug PFD group also showed a certain degree of fibrosis, and both the RLX-free and RLX@PMs groups showed a near-normal lung structure. In addition, according to the weight change curves of mice (FIG. 5), the mice both in the PBS and PFD groups showed progressive weight loss, and even all the mice in the PFD group died within three weeks. We noticed that the lung wet weights of mice in the RLX@PMs treatment group were significantly lower than those of mice in the PBS treatment group at week 4 (p<0.01) (FIG. 6). Compared with the free drug administered frequently, in the bleomycin-induced mouse model, a single inhalation of RLX@PMs had a better effect on restoring lung structure. In summary, these data show that bleomycin treatment leads to obvious pulmonary fibrosis and impaired lung function, while inhalation of RLX@PMs can prevent the progression of bleomycin-induced pulmonary fibrosis and preserve lung function. These results strongly suggest that in the case of long-term continuous dosing, RLX has excellent clinical translational potential in the treatment of IPF when administered continuously for a long time.

### 2.5.3 Examination Results of Hematoxylin and Eosin, Masson Trichrome and Sirius Red Staining

The degree of airway inflammation and tissue damage was evaluated by examining lung histopathology. Chronic exposure of mice to bleomycin was shown to increase airway remodeling, angiogenesis and parenchymal inflammation, but treatment with PFD or RLX attenuated these effects. As shown by the H&E staining results in FIG. 7a, in the bleomycin-induced fibrosis mouse model, the accumulation of inflammatory cells in the peribronchial and perivascular spaces of mice inhaling PBS increased significantly, but it decreased significantly in the lung sections of mice receiving RLX@PMs. Consistent with these results, when Masson trichromatic staining was used to analyze the fibrosis around bronchi (FIGS. 7b and 9), it was found that the collagen accumulation of mice inhaling RLX or PFD was lower than that of the PBS group. The amount of collagen deposition in the RLX@PMs group was less, indicating improved lung function.

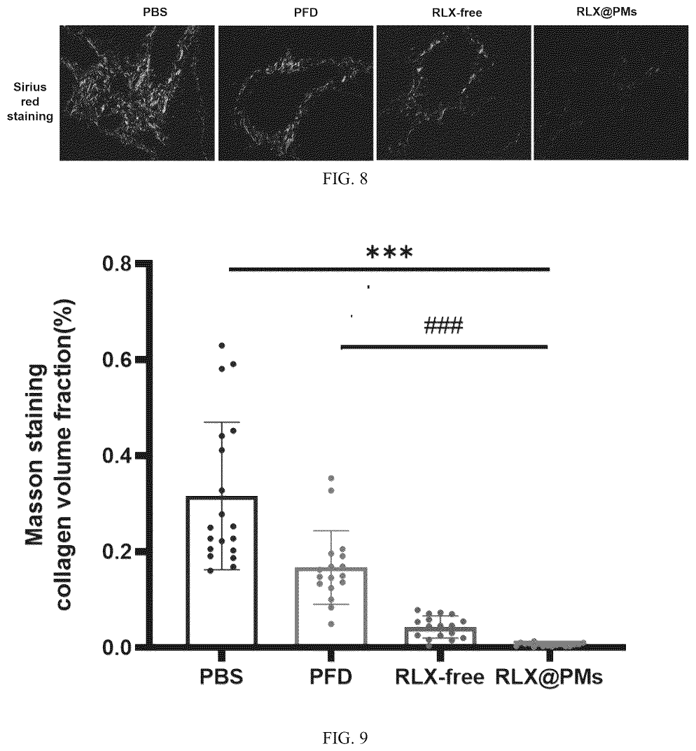

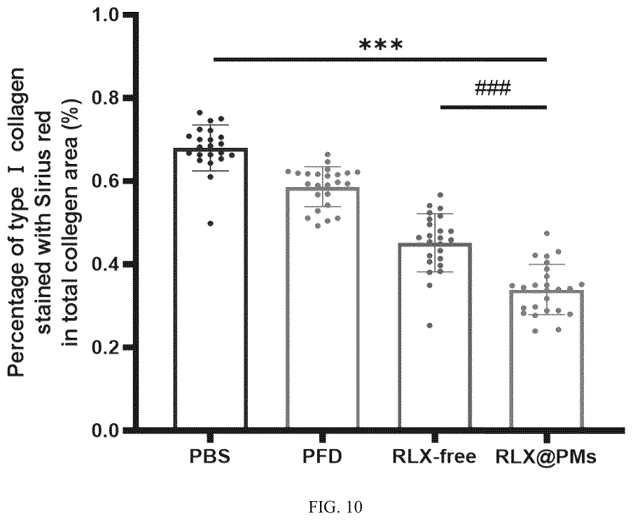

Under the polarization microscope, type 1 collagen fibers are tightly arranged red fibers, showing strong birefringence; type III collagen fibers have sparse network structure, showing weak birefringence, and they are tiny green fibers. As shown in FIG. 8 and FIG. 10, Sirius red staining showed that there was obvious collagen deposition in the lung tissue of IPF mice, and the positive control mice showed slight therapeutic effect. The results show that RLX-free or RLX@PMs treatment effectively inhibits the disease progression in mice with pulmonary fibrosis.

1. A preparation method for inhalable porous microspheres loaded with recombinant human relaxin-2, comprising the steps of:
(1) dissolving recombinant human relaxin-2 (RLX) in PBS containing bovine serum albumin to serve as an internal aqueous phase, and dissolving a carrier material ester-terminated polylactic acid copolymer OH-PLGA75/25-COOR in dichloromethane to serve as an oil phase; adding the internal aqueous phase into the oil phase, and emulsifying by high-speed shearing at 10,000 rpm for 2 minutes to form a water-in-oil (W1/O) emulsion; using 20% (v/v) polyvinyl alcohol (PVA) solution as an external aqueous phase;
(2) injecting the water-in-oil (W1/O) emulsion obtained in step (1) into a 20% (v/v) PVA solution pre-cooled in an ice bath, mixing and emulsifying for 2 minutes by using high-speed shearing force to form an oil-in-water (O/W2) emulsion, stirring the emulsion in an ice bath to evaporate DCM, collecting non-porous microspheres by centrifuging at a speed of 5,000-8,000 rpm for 5-8 minutes, washing the microspheres with deionized water pre-cooled in an ice bath, and collecting all precipitates to obtain non-porous microspheres loaded with RLX (RLX@SMs); and
(3) preparing the porous microspheres loaded with recombinant human relaxin-2 (RLX@PMs) from the non-porous microspheres loaded with RLX obtained in step (2) by programmed cooling which specifically comprises the steps of: first, freezing the collected non-porous microspheres at −20° C. for 4-6 hours, then immediately transferring them to a −80° C. freezer and maintaining for 8 hours, and finally lyophilizing the microspheres in a freeze dryer for 24 hours, to obtain the porous microspheres;
in step (1), the recombinant human relaxin-2 (RLX) is dissolved in PBS containing 0.1% (v/w) of bovine serum albumin;
in step (1), the ester-terminated polylactic acid copolymer OH-PLGA75/25-COOR has LA:GA=75:25 and a molecular weight (Mw) of 28,500 Da;
in step (1), the carrier material ester-terminated polylactic acid copolymer OH-PLGA75/25-COOR is dissolved in DCM at a concentration of 100 mg/mL to serve as the oil phase;
the particle size of the porous microspheres obtained in step (3) is 8-10 μm, and the particle density and porosity are 0.199±0.041 g·cm⁻³ and 95.0±0.8%, respectively.

2. The preparation method according to claim 1, characterized in that, in step (2), the oil-in-water (O/W2) emulsion is stirred in ice water to evaporate DCM, and the non-porous microspheres are collected by centrifugation at 8,000 rpm for 5 minutes.

3. The preparation method according to claim 1, characterized in that, in step (2), the collected non-porous microspheres are washed three times with deionized water pre-cooled in an ice bath.

4. The preparation method according to claim 1, characterized in that, in step (3), the collected non-porous microspheres are first frozen at −20° C. for 4 hours.

5. An inhalable porous microsphere loaded with recombinant human relaxin-2 prepared by the preparation method according to claim 1.

6. An inhalable porous microsphere loaded with recombinant human relaxin-2 prepared by the preparation method according to claim 2.

7. An inhalable porous microsphere loaded with recombinant human relaxin-2 prepared by the preparation method according to claim 3.

8. An inhalable porous microsphere loaded with recombinant human relaxin-2 prepared by the preparation method according to claim 4.
